(19) United States
(12) Patent Application Publication
(10) Pub. No.: US 2024/0358659 A1
(43) Pub. Date: Oct. 31, 2024

(54) Uses of Propranolol for Managing Viral Infections

(71) Applicants: Emory University, Atlanta, GA (US); Leiden University Medical Center, Leiden (NL)

(72) Inventors: Jack L. Arbiser, Atlanta, GA (US); Martijn J. van Hemert, Leiden (NL); Melissa Thaler, Leiden (NL)

(21) Appl. No.: 18/685,765

(22) PCT Filed: Aug. 30, 2022
(86) PCT No.: PCT/US2022/042011

§ 371 Date: Feb. 22, 2024

Related U.S. Application Data
(60) Provisional application No. 63/238,619, filed on Aug. 30, 2021.

(51) Int. Cl. A61K 31/138; A61P 31/14

(52) U.S. Cl. CPC A61K 31/138; A61P 31/14

(57) ABSTRACT

This disclosure relates to methods of treating or preventing a viral infection, such as a coronavirus or SARS-CoV-2 infection, comprising administering an effective amount of propranolol, prodrugs, or salts thereof to a subject in need thereof. In certain embodiments, propranolol is the R-propranolol isomer in a pharmaceutical composition comprising R-propranolol in enantiomeric excess.

### CROSS-REFERENCE TO RELATED APPLICATIONS

This application claims the benefit of U.S. Provisional Application No. 63/238,619 filed Aug. 30, 2021. The entirety of this application is hereby incorporated by reference for all purposes.

### BACKGROUND

Some common colds are due to certain coronavirus (CoV) strains associated with mild symptoms. More dangerous human strains such as severe acute respiratory syndrome associated coronavirus (SARS-CoV-1) and SARS-CoV-2 (also referred to as COVID-19) are believed to result from coronavirus strains jumping to humans by secondary zoonotic transfers. In humans, SARS-CoV-2 can be transferred from individuals who have mild symptoms or are asymptomatic and has caused numerous deaths worldwide. Thus, there is a need to identify improved therapies.

Propranolol was traditionally characterized as a beta-blocker and used clinically for treating hypertension, atrial fibrillation, and preventing a heart attack. Pharmaceutical compositions of propranolol are a racemic mixture of two enantiomers, R(+) and S(−), e.g. Inderal® (+)-1-[(1-methylethyl)amino]-3-(1-naphthalenyloxy)-2-propanol hydrochloride. As beta-blockers alter functions of the heart and circulatory system, side effects include bradycardia (slow heart rate) and hypotension (low blood pressure). Co-administration of certain drugs with propranolol are reported to lead to clinically relevant drug interactions and changes in efficacy and/or toxicity. Thus, avoiding these side effects are desirable.

Sasaki et al. report propranolol exhibits activity against hemangiomas independent of beta blockade. npj Precis Onc, 3, 27 (2019).

Overman et al. report R-propranolol is a small molecule inhibitor of the SOX18 transcription factor in a rare vascular syndrome and hemangioma. Elife, 2019, 8:e43026.

References cited herein are not an admission of prior art.

### SUMMARY

This disclosure relates to methods of treating or preventing a viral infection, such as a coronavirus or SARS-CoV-2 infection, comprising administering an effective amount of propranolol, prodrugs, or salts thereof to a subject in need thereof. In certain embodiments, propranolol is the R-propranolol isomer in a pharmaceutical composition comprising R-propranolol in enantiomeric excess.

In certain embodiments, propranolol is a composition comprising the R or S enantiomer in enantiomeric excess or a racemic mixture of R and S propranolol. In certain embodiments, propranolol is a composition comprising R-propranolol with enantiomeric excess of greater than 60%, 70%, 80%, 90%, 95%, 97%, 98% or 99%. In certain embodiments, propranolol is a composition comprising S-propranolol with enantiomeric excess of greater than 60%, 70%, 80%, 90%, 95%, 97%, 98% or 99%.

In certain embodiments, propranolol is administered in combination with another antiviral agent. In certain embodiments, the agent is remdesivir, chloroquine, hydroxychloroquine, azithromycin, ivermectin, lopinavir, ritonavir, nitazoxanide, or combinations thereof.

In certain embodiments, the subject is more than 55, 65, or 75 years old. In certain embodiments, the subject is diagnosed with a severe acute infection requiring intensive care.

In certain embodiments, propranolol is in a pharmaceutical composition in the form of a table, pill, capsule, or gel. In certain embodiments, propranolol is in a pharmaceutical composition in the form or a liquid comprising pH buffering agents and optionally salts and/or saccharide or polysaccharide.

In certain embodiments, this disclosure relates to the production of a medicament for uses in treating or preventing a viral infection, such as a coronavirus or SARS-CoV-2 infection.

### BRIEF DESCRIPTIONS OF THE SEVERAL VIEWS OF THE DRAWINGS

FIG. 1A shows cytotoxicity data of R-propranolol and S-propranolol in Vero E6 cells in a 24 h assay. The results indicate R- and S-propranolol cause no significant cytotoxicity in Vero E6 cells up to 150 uM.

FIG. 1B shows cytotoxicity data of R-propranolol and S-propranolol in Calu cells in a 24 h assay. The results indicate R- and S-propranolol cause no significant cytotoxicity in Calu-HTB-55 cells up to 150 uM.

FIG. 2 shows inhibition data of SARS-CoV-2 replication in Vero E6 cells measured in CPE reduction assay. The 50% inhibitory concentration in cytopathic effect reduction assay in Vero E6 cells for R-propranolol is 12.25 uM and for S-propranolol is 15.34 uM. No cytotoxicity is observed at these concentrations. It is encouraging to see that R-propranolol, for which less side effects in patients are expected, has an even slightly better EC₅₀.

FIG. 3 shows data on the reduction of infectivity after incubation with R-propranolol for 1 hr at 37° C. was measure by plaque assay. This data indicates the antiviral effect of R-propranolol observed in cell-based assays is not caused by virucidal activity because incubation of SARS-CoV-2 in the presence of R-propranolol does not reduce infectivity of virus particles.

FIG. 4A shows inhibition data of SARS-CoV-2 replication by R-propranolol in viral load reduction assay in Vero E6 cells, determined by quantifying the number of released genome copies in the medium of infected cells by internally controlled multiplex TaqMan RT-qPCR in two independent experiments. This data indicates a viral load reduction assay in Vero E6 cells of about a 2.5 log reduction in extracellular SARS-CoV-2 genome copies after treatment of infected cells (MOI 1) with 50 uM R-propranolol when samples were analyzed at 16 h post-infection (pi). At 25 uM the R-propranolol has approximately the same efficacy as remdesivir.

FIG. 4B shows viral load reduction assay data in human lung epithelial (Calu) cells revealed about 1.5 log reduction in extracellular SARS-CoV-2 genome copies after treatment of infected cells (MOI 1) with 150 uM R-propranolol when samples were analyzed at 16 h pi.

FIG. 4C shows inhibition data of SARS-CoV-2 replication by R-propranolol in viral load reduction assay in Calu cells, determined by quantifying the number of released genome copies in the medium of infected cells by internally controlled multiplex TaqMan RT-qPCR.

FIG. 5 shows inhibition data of SARS-CoV-2 replication by R-propranolol in time of addition assay in Vero cells, determined by quantifying the number of infectious particles by plaque assay at 16 h pi. Time of addition assay of R-propranolol on Vero cells shows the highest reduction of viral load upon pre-treatment with R-propranolol.

FIG. 6 shows inhibition data of SARS-CoV-2 replication by R-propranolol on human airway epithelial cells determined by quantifying the number of released genome copies in the medium of infected cells by internally controlled multiplex TaqMan RT-qPCR.

FIG. 7 shows inhibition data of SARS-CoV-2 replication by R-propranolol on human airway epithelial (HAE) cells determined by quantifying the number of infectious particles by plaque assay at 72 h pi.

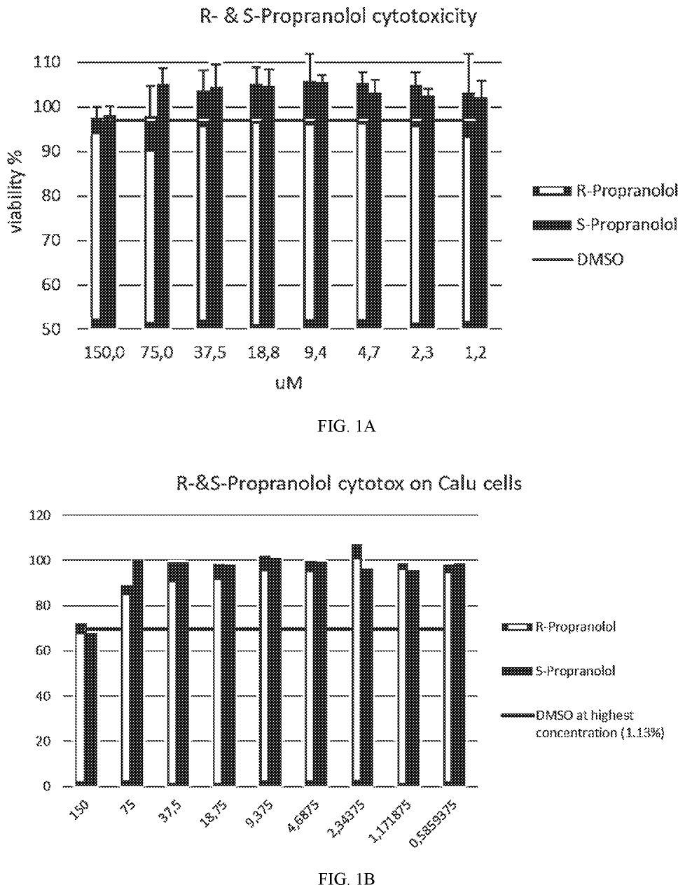

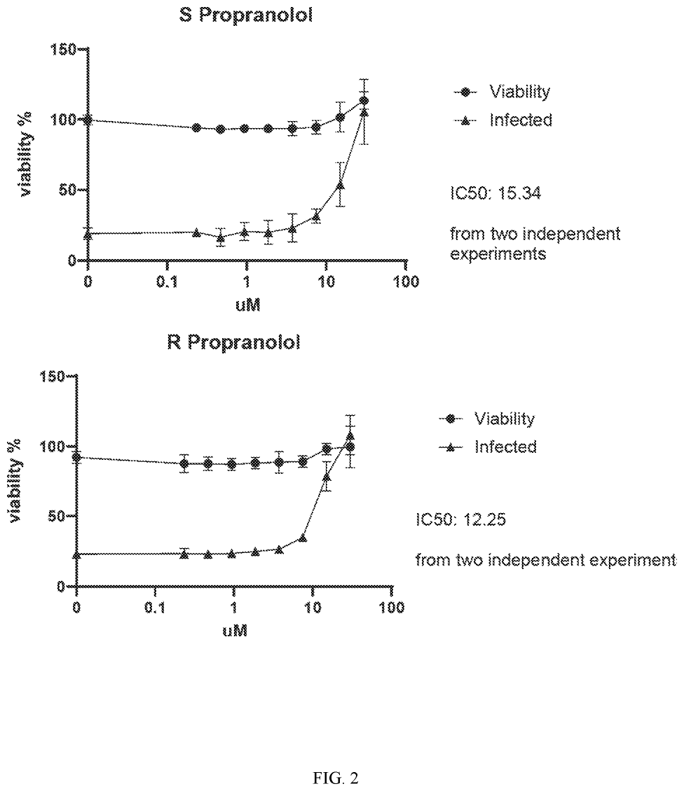

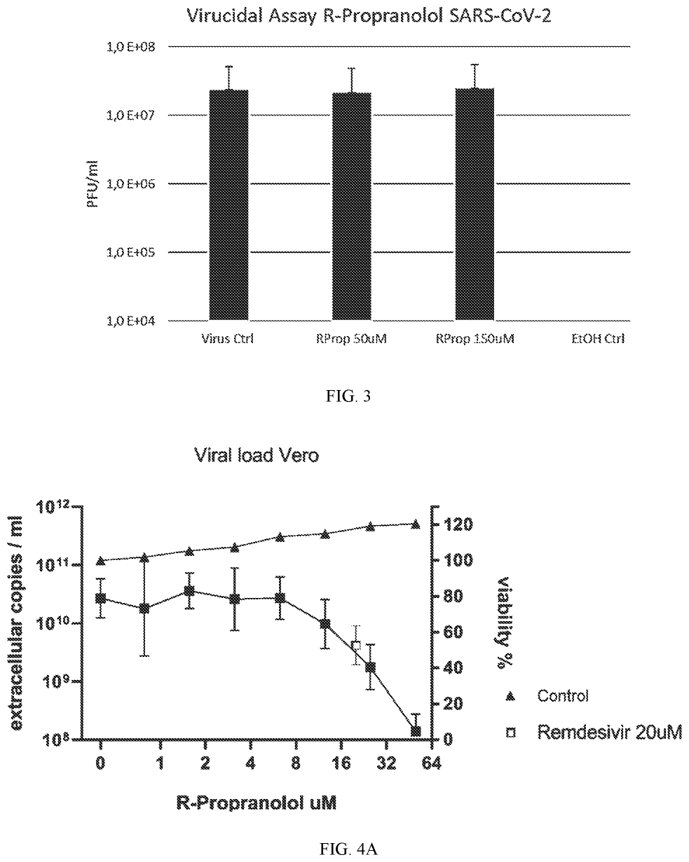

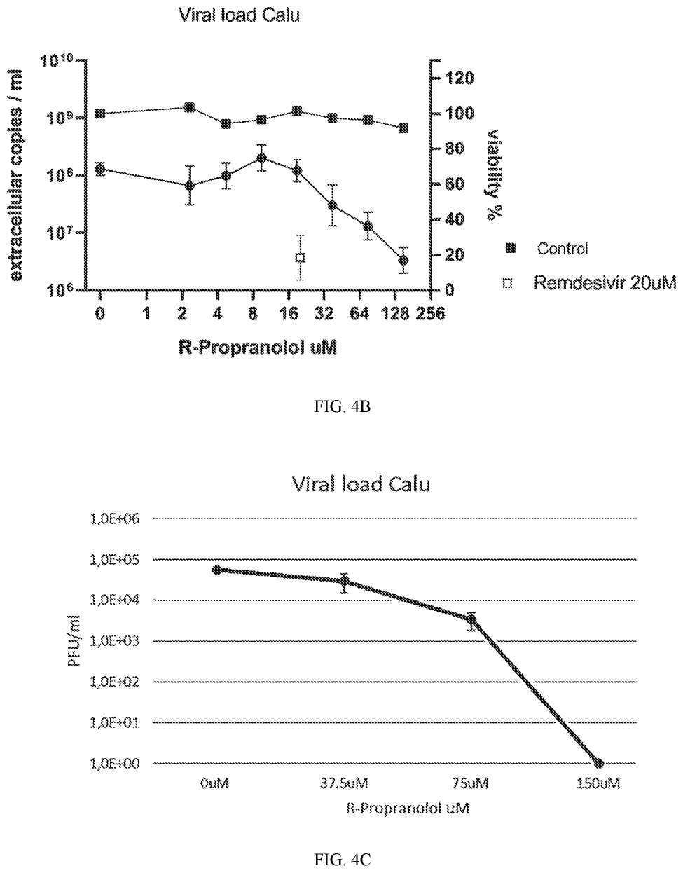

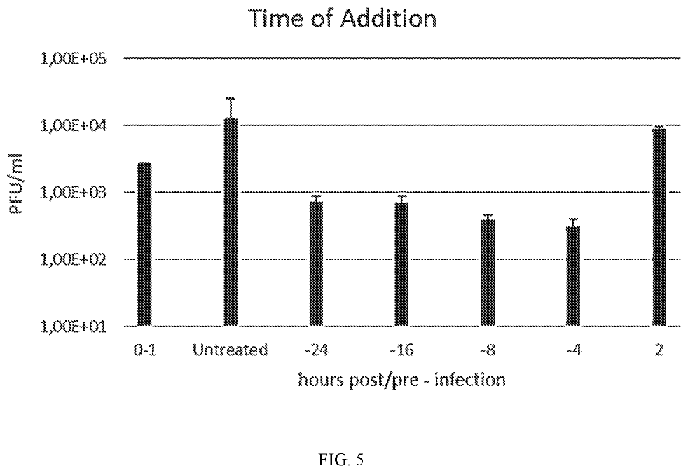

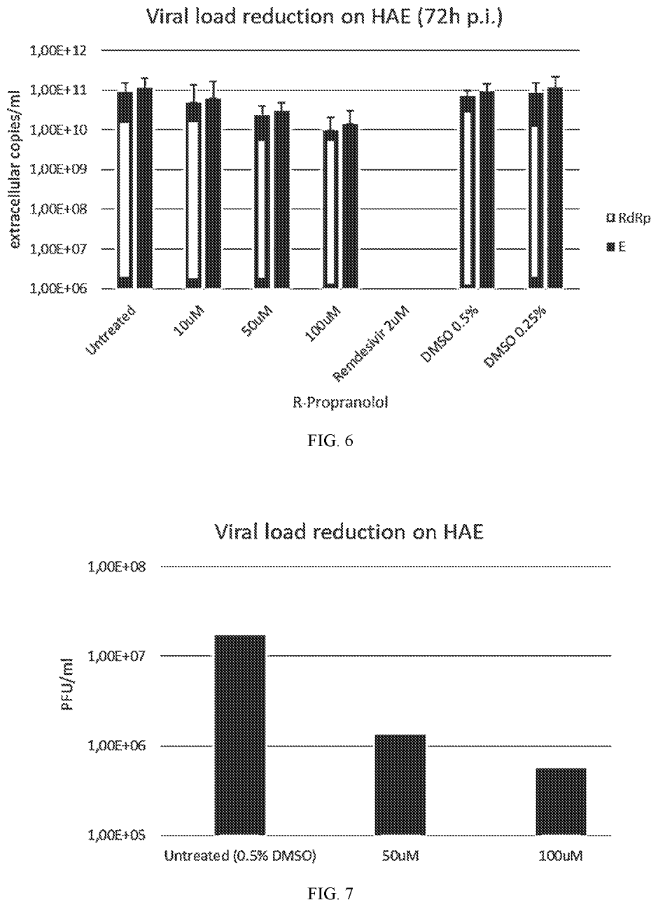

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. An “embodiment” of this disclosure refers to an example and infers that the example is not necessarily limited to the example.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic acceptable salts including the quaternary ammonium salts of the parent compound formed, and non-toxic inorganic or organic acids.

Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in compositions over the parent drug. A prodrug may be converted into the parent drug by various mechanisms, including enzymatic processes and metabolic hydrolysis. Typical prodrugs are esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol, i.e., hydroxy group.

### Methods of Use

This disclosure relates to methods of treating or preventing a viral infection, such as a coronavirus, severe acute respiratory syndrome coronavirus 1 (SARS-CoV-1), SARS-CoV-2 (COVID-19), middle east respiratory syndrome coronavirus (MERS-CoV), HCoV-229E, HCoV-NL63, HCoV-OC43, HCoV-HKU1, comprising administering an effective amount of propranolol, prodrugs, or salts thereof to a subject in need thereof.

In certain embodiments, propranolol is administered in combination with another antiviral agent. In certain embodiments, the agent is remdesivir, chloroquine, hydroxychloroquine, azithromycin, ivermectin, lopinavir, ritonavir, nitazoxanide, or combinations thereof.

In certain embodiments, the subject is diagnosed with a severe acute infection requiring intensive care. In certain embodiments, the subject is any age, e.g., less than 25, 20, 15, or 10 years old or the subject is more than 55, 65, or 75 years old.

In certain embodiments, this disclosure relates to treating or preventing chronic acute respiratory syndrome or associated side effects due to a coronavirus or other viral infections comprising administering a composition comprising an effective amount of propranolol to a subject in need thereof. In certain embodiments, the subject is diagnosed with a viral infection that poses a risk of developing an acute respiratory syndrome or chronic acute respiratory syndrome such as subject diagnosed with a high-risk coronavirus infection, e.g., SARS-CoV-1 or SARS-CoV-2 infection.

In certain embodiments, the composition administered daily. In certain embodiments, the composition is administered daily for more than 3, 5, 7 days or two weeks.

In certain embodiments, the compositions are administered daily, up to 2 times a day, 3 times a day, or as a continuous infusion. In certain embodiments, compositions are administered as a continuous enteral feeding for severely ill and/or hospitalized subjects.

In certain embodiments, the subject is diagnosed with fatigue, shortness of breath, anxiety, depression, brain fog, joint pain, and/or chest pain, and optionally diabetes, stroke, heart rhythm abnormality, and/or blood clot in the lungs.

In certain embodiments, the subject is more than 55, 65, or 75 years old and/or diagnosed with a severe acute infection requiring intensive care, pre-existing respiratory illness, obesity, diabetes, high blood pressure, chronic cardiovascular disease, chronic kidney disease, organ transplant, or cancer.

Although embodiments of this disclosure contemplate treatment of coronavirus infections, management of other viral infections are contemplated such as influenza virus, rhinovirus, hepatitis A, hepatitis B, hepatitis C, human papillomaviruses, human immunodeficiency, herpes virus, Epstein-Barr virus, herpes simplex virus, varicella-zoster virus, shingles virus, mumps virus, measles virus, West Nile virus, poliovirus, non-poliovirus enterovirus, respiratory syncytial virus, and parainfluenza virus.

In certain embodiments, propranolol is administered in combination with another anti-viral agent such as, abacavir, acyclovir, adefovir, amantadine, arbidol, baloxavir, boceprevir, daclatasvir, docosanol, edoxudine, enfuvirtide, famciclovir, foscarnet, ganciclovir, ibacitabine, idoxuridine, imiquimod, imunovir, lamivudine, letermovir, marboxil, methisazone, moroxydine, nexavir, oseltamivir, peramivir, penciclovir, pleconaril, ribavirin, rimantadine, simeprevir, sofosbuvir, taribavirin, telbivudine, tenofovir, trifluridine, tromantadine, umifenovir, valaciclovir, vidarabine, zanamivir, zidovudine, or combinations thereof.

### Pharmaceutical Compositions

Pharmaceutical compositions typically comprise an effective amount of compounds and a suitable pharmaceutical acceptable excipient or carrier. The preparations can be prepared in a manner known per se, which usually involves mixing the compounds according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. Pharmaceutically acceptable salts, solvates, and hydrates of the compounds listed are also useful in the method of the disclosure and in pharmaceutical compositions of the disclosure.

In certain embodiments, a composition comprising a (R)-(+)-1-(isopropylamino)-3-(naphthalen-1-yloxy)propan-2-ol in enantiomeric excess or (S)-(−)-1-(isopropylamino)-3-(naphthalen-1-yloxy)propan-2-ol in enantiomeric excess or racemic mixture or salt thereof as reported herein of the present disclosure can be administered to a subject either alone or as a part of a pharmaceutical composition.

In certain embodiments, propranolol is a composition comprising the R or S enantiomer in enantiomeric excess or a racemic mixture of R and S propranolol. In certain embodiments, the R isomer may be present in a composition with enantiomeric excess of greater than 60%. In certain embodiments, the R isomer may be present in enantiomeric excess of greater than 70%. In certain embodiments, the R isomer may be present in enantiomeric excess of greater than 80%. In certain embodiments, the R isomer may be present in enantiomeric excess of greater than 90%. In certain embodiments, the R isomer may be present in enantiomeric excess of greater than 95%. In certain embodiments, the S isomer may be present in a composition with enantiomeric excess of greater than 60%. In certain embodiments, the S isomer may be present in enantiomeric excess of greater than 70%. In certain embodiments, the S isomer may be present in enantiomeric excess of greater than 80%. In certain embodiments, the S isomer may be present in enantiomeric excess of greater than 90%. In certain embodiments, the S isomer may be present in enantiomeric excess of greater than 95%.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a tablet, pill, capsule, powders, granules, gel, gel capsule, or cream. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia gum, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof.

Solid dosage forms can be prepared with coatings and shells, such as enteric coatings and others well known in the art. They may contain opacifying agents and can also be of such composition that they release the active compound or compounds in a certain part of the intestinal tract in a delayed manner. In certain embodiments, pharmaceutical composition is in solid form surrounded by an enteric coating. In certain embodiments, the enteric coating comprises methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, or combinations thereof.

In certain embodiments, this disclosure contemplates an intravenous formulation with pH buffering agents and tonicity in a range representing physiological values (pH 7 to 8) or for bolus administration, e.g., containing normal saline or dextrose optionally containing pH buffering agents. In certain embodiments, the pharmaceutical composition is in the form of a sterilized pH buffered aqueous salt solution or a saline phosphate buffer between a pH of 6 to 8, optionally comprising a saccharide or polysaccharide.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

The pharmaceutical compositions of the present disclosure can be administered to subjects either topically to the skin, orally, rectally, parenterally (intravenously, intramuscularly, or subcutaneously), intracisternally, intravaginally, intraperitoneally, intravesically, locally (powders, ointments, or drops), or as a buccal or nasal spray.

In certain embodiments, the pharmaceutical compositions are in a form for inhalation. In certain embodiments, the pharmaceutical composition comprises a compound disclosed herein and a propellant. In certain embodiments, an aerosolizing propellant is compressed air, ethanol, nitrogen, carbon dioxide, nitrous oxide, hydrofluoroalkanes (HFAs), or combinations thereof.

In certain embodiments, the disclosure contemplates a pressurized or unpressurized container comprising a compound herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

Pharmaceutical compositions typically comprise an effective amount of a composition comprising (R)-(+)-1-(isopropylamino)-3-(naphthalen-1-yloxy)propan-2-ol in enantiomeric excess or (S)-(−)-1-(isopropylamino)-3-(naphthalen-1-yloxy)propan-2-ol in enantiomeric excess or racemic mixture or salt thereof as reported herein of the present disclosure and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet, 2006, 21(3):173-85, entitled “Human carboxylesterase isozymes: catalytic properties and rational drug design.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

1. A method of treating a viral infection comprising administering an effective amount of propranolol, prodrugs, or salts thereof to a subject in need thereof.

2. The method of claim 1, wherein the viral infection is a coronavirus.

3. The method of claim 1, wherein the viral infection is SARS-CoV-2

4. The method of claim 1, wherein propranolol is a composition comprising a racemic mixture of R and S propranolol.

5. The method of claim 1, wherein propranolol is a composition comprising R-propranolol with enantiomeric excess of greater than 90%.

6. The method of claim 1, wherein propranolol is a composition comprising S-propranolol with enantiomeric excess of greater than 90%.

7. The method of claim 1, wherein propranolol is administered in combination with another antiviral agent.

8. The method of claim 1, wherein the subject is more than 55 years old.

9. The method of claim 1, wherein the subject is diagnosed with a severe acute infection requiring intensive care.

10. The method of claim 1, wherein the propranolol a pharmaceutical composition in the form of a table, pill, capsule, or gel.

11. The method of claim 1, wherein the propranolol is a pharmaceutical composition in the form or a liquid comprising pH buffering agents and optionally salts and/or sugar.
